(19) United States
(12) Patent Application Publication
(10) Pub. No.: US 2022/0306581 A1
(43) Pub. Date: Sep. 29, 2022

(54) ANTIBACTERIAL MONOMER, ANTIBACTERIAL POLYMER COMPOSITION INCLUDING THE ANTIBACTERIAL MONOMER, ANTIBACTERIAL FILM OBTAINED FROM THE ANTIBACTERIAL POLYMER COMPOSITION, AND ARTICLE INCLUDING THE ANTIBACTERIAL FILM

(71) Applicant: Samsung Electronics Co., Ltd., Suwon-si (KR)

(72) Inventors: Wonsuk CHANG, Hwaseong-si (KR); Inki KIM, Hwaseong-si (KR); Ginam KIM, Seongnam-si (KR)

(21) Appl. No.: 17/545,060

(22) Filed: Dec. 8, 2021

(30) Foreign Application Priority Data
Mar. 26, 2021 (KR) 10-2021-0039997

(51) Int. Cl. C07D 213/74; A01N 43/40; C07D 213/75; A01N 25/10; C08F 220/12

(52) U.S. Cl. CPC C07D 213/74; A01N 43/40; C07D 213/75; A01N 25/10; C08F 220/12

(57) ABSTRACT

A hard coating film including a metal-free acrylate-based resin and having a water contact angle of about 40° or less and a pencil surface hardness of about 6H or more at a load of 1 kg; a composition for hard coating used in preparation of the hard coating film; a laminate including the hard coating film, a method of preparing the laminate; and an article including the laminate.

### CROSS-REFERENCE TO RELATED APPLICATION

This application is based on and claims priority to Korean Patent Application No. 10-2021-0039997, filed on Mar. 26, 2021, in the Korean Intellectual Property Office, and all the benefits accruing therefrom under 35 U.S.C. § 119, the entire content of which is incorporated by reference herein.

### BACKGROUND

### 1. Field

The present disclosure relates to an antibacterial monomer having a novel structure, an antibacterial polymer composition including the antibacterial monomer, an antibacterial film obtained from the antibacterial polymer composition, and an article including the antibacterial film.

### 2. Description of the Related Art

When a surface of a substrate frequently used in daily life, such as glass or plastic, is contaminated from the outside, clean water is needed for washing the surface. Also, the more severe the contamination of the surface of the substrate, the greater the amount of washing water needed.

In recent years, the number of available water resources are decreasing due to environmental pollution and changes in climate, and studies on the supply of available water such as desalination and water recycling are being conducted in response to the water shortage. Still, there is a limit to solving the global water shortage.

When securing washing water is difficult due to the lack of water, an environment in which various bacteria thrive is created by contaminants, and the spread of bacteria through animals threatens the lives of many other animals.

Therefore, there is still a need for an antibacterial film having excellent surface hardness, and that is capable of suppressing bacterial growth as well as allowing easy cleaning of a contaminated surface, and a composition for preparing the antibacterial film.

### SUMMARY

Provided herein are an antibacterial film capable of suppressing bacterial propagation, an antibacterial polymer composition for preparation of the antibacterial film, and an article including the antibacterial film.

Additional aspects will be set forth in part in the description which follows and, in part, will be apparent from the description, or may be learned by practice of the presented embodiments of the disclosure.

According to an aspect of an embodiment, an antibacterial monomer is represented by Formula 1:

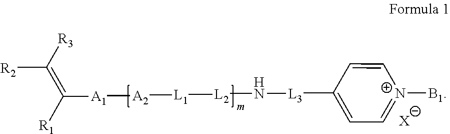

In Formula 1,
A₁ is *—C(═O)—*′, *—C(═O)O—*′, or *—C(O)NH—*′,
A₂ is *—O—*′, *—S—*′, or *—Se—*′,
L₁, L₂, and L₃ are each independently, a single bond, a substituted or unsubstituted C₁-C₂₀ alkylene group, or a substituted or unsubstituted C₂-C₂₀ alkenylene group,
m is an integer selected from 0 to 3,
B₁ is a substituted or unsubstituted C₁-C₂₀ alkyl group,
R₁ to R₃ are each independently, hydrogen, deuterium, —F, —Cl, —Br, —I, a hydroxyl group, a cyano group, an amino group, a nitro group, a substituted or unsubstituted C₁-C₂₀ alkyl group, a substituted or unsubstituted C₂-C₂₀ alkenyl group, a substituted or unsubstituted C₂-C₂₀ alkynyl group, or a substituted or unsubstituted C₁-C₂₀ alkoxy group,
X is a halogen anion, and
* and *′ each indicate a binding site to an adjacent atom.

According to an aspect of another embodiment, an antibacterial polymer composition includes the antibacterial monomer; a multifunctional monomer; and a monofunctional monomer with a hydrophilic group, different from the antibacterial monomer.

According to an aspect of another embodiment, an antibacterial film includes a polymer including at least one repeating unit represented by Formula 3:

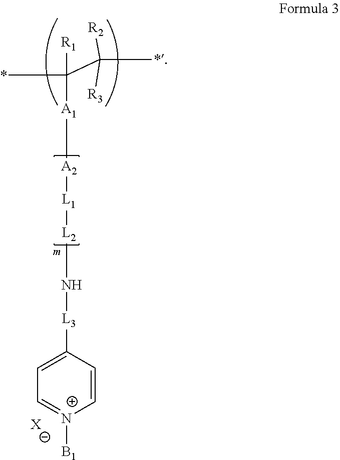

In Formula 3,
A₁ is *—C(═O)—*′, *—C(═O)O—*′, or *—C(O)NH—*′,
A₂ is *—O—*′, *—S—*′, or *—Se—*′,
L₁, L₂, and L₃ are each independently, a single bond, a substituted or unsubstituted C₁-C₂₀ alkylene group, or a substituted or unsubstituted C₂-C₂₀ alkenylene group,
m is an integer selected from 0 to 3,
B₁ is a substituted or unsubstituted C₁-C₂₀ alkyl group,
R₁ to R₃ are each independently, hydrogen, deuterium, —F, —Cl, —Br, —I, a hydroxyl group, a cyano group, an amino group, a nitro group, a substituted or unsubstituted C₁-C₂₀ alkyl group, a substituted or unsubstituted C₂-C₂₀ alkenyl group, a substituted or unsubstituted C₂-C₂₀ alkynyl group, or a substituted or unsubstituted C₁-C₂₀ alkoxy group,
X is a halogen anion, and
* and *′ each indicate a binding site to an adjacent atom.

According to an aspect of another embodiment, an article includes the antibacterial film.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic view of a laminate including an antibacterial film according to an embodiment;

FIG. 2 is a schematic view of a laminate including an antibacterial film according to an embodiment;

FIG. 3 is a schematic view of a laminate including an antibacterial film according to an embodiment;

FIG. 4 is a schematic view of a laminate including an antibacterial film according to an embodiment; and

FIG. 5 shows images of a result of an antibacterial test of Example 4.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

Various embodiments are shown in the accompanying drawings. This inventive concept may, however, be embodied in many different forms and should not be construed as limited to the embodiments set forth herein. Rather, these embodiments are provided so that this disclosure will be thorough and complete, and will fully convey the scope of the inventive concept to those skilled in the art. Like reference numerals in the drawings denote like elements.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third,” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings herein.

The terminology used herein is for the purpose of describing particular embodiments only and is not intended to be limiting the inventive concept. As used herein, the singular forms “a,” “an,” and “the” are intended to include the plural forms, including “at least one,” unless the content clearly indicates otherwise. “At least one” is not to be construed as limiting “a” or “an.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Spatially relative terms, such as “beneath,” “below,” “lower,” “above,” or “upper”, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” may encompass both an orientation of above and below. The device may be otherwise oriented (rotated 90 degrees or at other orientations) and the spatially relative descriptors used herein interpreted accordingly.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features Moreover, sharp angles that are illustrated may be rounded. Thus, the regions illustrated in the figures are schematic in nature and their shapes are not intended to illustrate the precise shape of a region and are not intended to limit the scope of the present claims.

While a particular embodiment has been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, 10%, or 5% of the stated value.

In any formula, * and *′ each indicate a binding site to a neighboring atom or a neighboring functional group.

The term “room temperature” used herein refers to a temperature of about 25° C.

Hereinafter, a composition for forming a hard coating film, a hard coating film, a method of preparing a hard coating film, and an article according to at least one example embodiment will be described in detail.

### Antibacterial Monomer

According to an aspect of an embodiment, an antibacterial monomer may be represented by Formula 1:

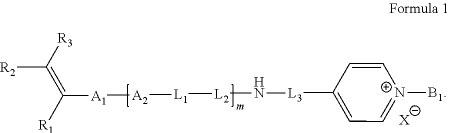

A₁ is *—C(═O)—*′, *—C(═O)O—*′, or *—C(O)NH—*′,
A₂ is *—O—*′, *—S—*′, or *—Se—*′,
L₁, L₂, and L₃ are each independently, a single bond, a substituted or unsubstituted C₁-C₂₀ alkylene group, or a substituted or unsubstituted C₂-C₂₀ alkenylene group,
m is an integer selected from 0 to 3,
B₁ is a substituted or unsubstituted C₁-C₂₀ alkyl group,
R₁ to R₃ are each independently, hydrogen, deuterium, —F, —Cl, —Br, —I, a hydroxyl group, a cyano group, an amino group, a nitro group, a substituted or unsubstituted C₁-C₂₀ alkyl group, a substituted or unsubstituted C₂-C₂₀ alkenyl group, a substituted or unsubstituted C₂-C₂₀ alkynyl group, or a substituted or unsubstituted C₁-C₂₀ alkoxy group,
X is a halogen anion, and
* and *′ each indicate a binding site to an adjacent atom.

In an embodiment, at least one substituent of the substituted C₁-C₂₀ alkylene group, substituted C₂-C₂₀ alkenylene group, substituted C₁-C₂₀ alkyl group, substituted C₂-C₂₀ alkenyl group, substituted C₂-C₂₀ alkynyl group, and substituted C₁-C₂₀ alkoxy group may be deuterium, —F, —Cl, —Br, —I, a hydroxyl group, a cyano group, a nitro group, an amino group, an amidino group, a hydrazine group, a hydrazone group, a C₁-C₂₀ alkyl group, a C₂-C₂₀ alkenyl group, a C₂-C₂₀ alkynyl group, or a C₁-C₂₀ alkoxy group.

In an embodiment, in Formula 1, m may be 0 or 1. When m is 0, A₁ may be —C(═O)—, and when m is 1, A₁ may be —C(═O)—, and A₂ may be —O—,

In an embodiment,
A₁ is *—C(═O)—*′, or *—C(O)NH—*′,
A₂ is *—O—*′,
L₁, L₂, and L₃ each independently, a single bond, or a substituted or unsubstituted C₁-C₂₀ alkylene group,
m is an integer selected from 0 to 2,
B₁ is a substituted or unsubstituted C₁-C₂₀ alkyl group,
R₁ to R₃ are each independently, hydrogen, deuterium, a substituted or unsubstituted C₁-C₂₀ alkyl group, a substituted or unsubstituted C₂-C₂₀ alkenyl group, a substituted or unsubstituted C₂-C₂₀ alkynyl group, or a substituted or unsubstituted C₁-C₂₀ alkoxy group,
X is a halogen anion, and
* and *′ each indicate a binding site to an adjacent atom.

In an embodiment,
A₁ is *—C(═O)—*′, or *—C(O)NH—*′,
A₂ is *—O—*′,
L₁, L₂, and L₃ each independently, a single bond, or a substituted or unsubstituted C₁-C₂₀ alkylene group,
m is an integer selected from 0 to 1,
B₁ is a substituted or unsubstituted C₁-C₂₀ alkyl group,
R₁ to R₃ are each independently, hydrogen, or a substituted or unsubstituted C₁-C₂₀ alkyl group,
X is a halogen anion, and
* and *′ each indicate a binding site to an adjacent atom.

In an embodiment, in Formula 1, L₁, L₂, and L₃ may be each independently a single bond or a group represented by Formula 2:

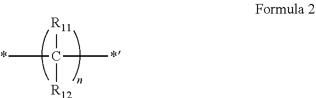

In Formula 2,
n is an integer selected from 1 to 20,
R₁₁ and R₁₂ are each independently hydrogen, deuterium, —F, —Cl, —Br, —I, a hydroxyl group, a cyano group, an amino group, a nitro group, a polyether group, a C₁-C₂₀ alkyl group, or a C₁-C₂₀ alkoxy group, and
* and *′ each indicate a binding site to an adjacent atom.

For example, L₁, L₂, and L₃ may be each independently a single bond or a group of Formulae 2-1 to 2-10:

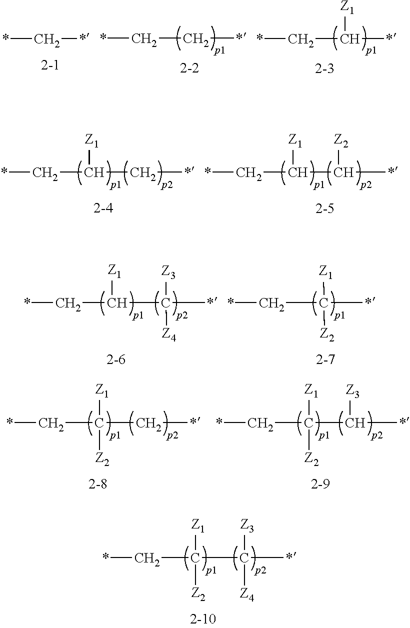

In Formulae 2-1 to 2-10,
p1 is an integer selected from 1 to 19,
p2 is an integer selected from 1 to 18,
when p1 and p2 are concurrently present, the sum of p1 and p2 is an integer of 19 or less,
Z₁ to Z₄ are each independently a hydroxyl group, an amino group, a nitro group, or a polyether group, and
* and *′ each indicate a binding site to an adjacent atom.

In an embodiment, L₃ may be a single bond.

In an embodiment, L₁ and L₂ may be groups represented by Formula 2, wherein, in Formula 2, at least one of R₁₁ and R₁₂ may be a hydroxyl group, an amino group, a nitro group, or a polyether group.

In an embodiment, in Formula 1, B₁ may be an unsubstituted C₁-C₂₀ alkyl group.

In an embodiment, in Formula 1, B₁ may be a straight-chain C₁-C₂₀ alkyl group, which may be, for example, a methyl group, an ethyl group, a propyl group, an n-propyl group, an isopropyl group, an n-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, or an n-eicosyl group.

In an embodiment, in Formula 1, R₁ to R₃ may be each independently hydrogen, deuterium, —F, —Cl, —Br, —I, a hydroxyl group, a cyano group, an amino group, a nitro group, a C₁-C₂₀ alkyl group, or a C₁-C₂₀ alkoxy group; or a C₁-C₂₀ alkyl group or a C₁-C₂₀ alkoxy group, each substituted with at least one deuterium, —F, —Cl, —Br, —I, a hydroxyl group, a cyano group, an amino group, a nitro group, or a C₁-C₂₀ alkoxy group.

In an embodiment, R₁ may be hydrogen, deuterium, —F, —Cl, —Br, —I, a hydroxyl group, a cyano group, an amino group, a nitro group, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group; and a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group, each substituted with at least one of deuterium, —F, —Cl, —Br, —I, a hydroxyl group, a cyano group, an amino group, a nitro group, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group.

In an embodiment, R₂ and R₃ may be each independently hydrogen, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group.

In an embodiment, R₁ to R₃ are each independently hydrogen, an unsubstituted C₁-C₆ alkyl group, an unsubstituted C₂-C₆ alkenyl group, an unsubstituted C₂-C₆ alkynyl group, or a substituted or an unsubstituted C₁-C₆ alkoxy group. For example, R₂ and R₃ may each be hydrogen, and R₁ may be hydrogen or a methyl group.

In an embodiment, the antibacterial monomer may be Compound 1, Compound 2, or Compound 3:

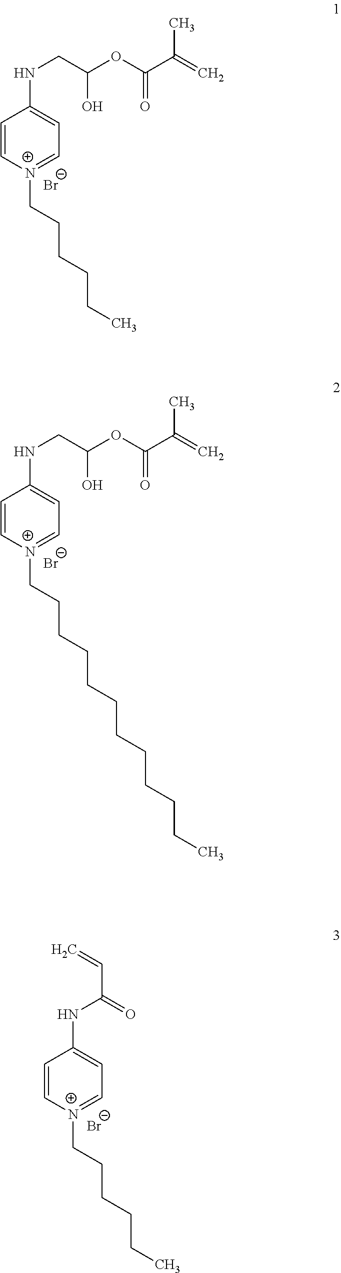 [SMILES: C=C(C)C(=O)OC(O)CNc1cc[n+](CCCCCC)cc1 ; [Br-] ; C=C(C)C(=O)OC(O)CNc1cc[n+](CCCCCCCCCCCC)cc1 ; [Br-] ; C=CC(=O)Nc1cc[n+](CCCCCC)cc1 ; [Br-]]

Since the antibacterial monomer represented by Formula 1 may have a double bond at the terminal end thereof, a film may be rapidly cured by UV irradiation, and since -A₂-L₁-L₂- contains a hydrophilic group, hydrophilicity may be imparted to the cured film. In an embodiment, pyridinium cations may capture bacteria, and the N-alkyl group of pyridinium may penetrate into the cell membrane of the bacteria and elute the cytoplasm, thereby killing the bacteria.

In an embodiment, when an acryl group is located at the para position of pyridinium in the antibacterial monomer represented by Formula 1, the film may be oriented perpendicular to a substrate during crosslinking to the substrate, and thus the film may provide better antibacterial properties to the substrate.

In an embodiment, the antibacterial monomer represented by Formula 1 may be polymerized with other polymer monomers to impart antibacterial properties to the film.

### Antibacterial Polymer Composition

According to an aspect of an embodiment, an antibacterial polymer composition may include the antibacterial monomer described herein; a multifunctional monomer; and a monofunctional monomer with a hydrophilic group, different from the antibacterial monomer. One or more other monofunctional monomers that are different from the antibacterial monomer, the monofunctional monomer with a hydrophilic group, and a monofunctional monomer without a hydrophilic group may optionally be present. The one or more other optional monofunctional monomers different from the antibacterial monomer and the monofunctional monomer with a hydrophilic group, and a monofunctional monomer without a hydrophilic group may be referred to herein as an “monofunctional optional monomer” for convenience.

In an embodiment, an amount of the antibacterial monomer may be about 0.1 weight % (wt %) or more based on the total amount of monomers in the antibacterial polymer composition.

For example, the amount of the antibacterial monomer may be in a range of about 0.1 wt % to about 3 wt %, about 0.1 wt % to about 2.80 wt %, about 0.1 wt % to about 2.60 wt %, about 0.1 wt % to about 2.40 wt %, about 0.1 wt % to about 2.20 wt %, about 0.1 wt % to about 2.0 wt %, about 0.1 wt % to about 1.8 wt %, about 0.1 wt % to about 1.6 wt %, about 0.1 wt % to about 1.4 wt %, about 0.1 wt % to about 1.0 wt %, or about 0.15 wt % to about 2.95 wt %, based on the total amount of monomers in the antibacterial polymer composition.

The antibacterial polymer composition including the antibacterial monomer is effective in killing bacteria by having the pyridinium alkyl chain on a surface thereof derived from the antibacterial monomer during curing of the composition.

Details of the antibacterial monomer may be referred to the description above.

In an embodiment, the multifunctional monomer may be an (meth)acrylate-based (i.e., (meth)acrylate-containing) multifunctional monomer, and the monofunctional monomer may be an (meth)acrylate-based (i.e., (meth)acrylate-containing) monofunctional monomer. The monofunctional optional monomer may be an (meth)acrylate-based (i.e., (meth)acrylate-containing) monofunctional monomer.

For example, the multifunctional monomer may be a multifunctional acrylate monomer or a multifunctional methacrylate. For example, the monofunctional hydrophilic monomer may be a monofunctional acrylate or a monofunctional methacrylate. For example, the monofunctional optional monomer may be a monofunctional acrylate or a monofunctional methacrylate. In an embodiment, the polymeric double bond of the antibacterial monomer forms a rapid crosslinking bond with a multifunctional monomer and a monofunctional monomer (and a monofunctional optional monomer) under the UV light and thus may form a cured film.

In an embodiment, an amount of the multifunctional monomer may be greater than that of the total amount of monofunctional monomer (i.e., the combined amount of the monofunctional monomer with a hydrophilic group and the monofunctional optional monomer). For example, an amount of the monofunctional monomer with a hydrophilic group or total amount of monofunctional monomer may be in a range of about 29 wt % to about 45 wt % based on the total amount of the composition. For example, the amount of the monofunctional monomer with a hydrophilic group or the total amount of monofunctional monomer may be in a range of about 29 wt % to about 45 wt %, about 29 wt % to about 43 wt %, about 29 wt % to about 41 wt %, about 29 wt % to about 39 wt %, about 29 wt % to about 37 wt %, about 29 wt % to about 35 wt %, about 29 wt % to about 33 wt %, about 29 wt % to about 31 wt %, or about 29 wt % to about 30 wt % based on the total amount of the composition.

In an embodiment, a content ratio of the multifunctional monomer to the total monofunctional monomer may be in a range of about 1:1 to about 1.5:1. For example, the content ratio of the multifunctional monomer to the total monofunctional monomer may be in a range of about 1:1 to about 1.4:1, about 1:1 to about 1.3:1, about 1:1 to about 1.2:1, or about 1:1 to about 1.1:1.

The antibacterial polymer composition may simultaneously include a multifunctional monomer and a total monofunctional monomer at a content ratio of the multifunctional monomer to the monofunctional monomer in a range of about 1:1 to about 1.5:1, and thus when the antibacterial polymer composition is cured, a high hardness of the cured film may be achieved by crosslinking of the multifunctional monomer. In an embodiment, since the monofunctional monomer with a hydrophilic group may be included as a monofunctional monomer, the monofunctional monomer with a hydrophilic group may be crosslinked on one side of the multifunctional monomer and exposed to the outside, thereby forming an antibacterial polymer film with hydrophilicity imparted to the surface.

The multifunctional monomer, and the monofunctional monomer, and the monofunctional optional monomer (if present) may be an (meth)acrylate-based monomer, i.e., a monomer including an acrylate group, a methacrylate group, or a combination thereof, which may serve as a crosslinking group during the process of curing and thus may form a polymer film having a 3-dimensional net structure.

In an embodiment, the multifunctional monomer may be a hexaacrylate monomer, a pentaacrylate monomer, a tetraacrylate monomer, a triacrylate monomer, or a diacrylate monomer. For example, the multifunctional monomer may include a mixture of at least two of a hexaacrylate monomer, a pentaacrylate monomer, a tetraacrylate monomer, a triacrylate monomer, or a diacrylate monomer.

In an embodiment, the multifunctional monomer may include a mixture of a tetraacrylate monomer, a triacrylate monomer, and a diacrylate monomer.

In an embodiment, the multifunctional monomer may be pentaerythritol tetraacrylate, pentaerythritol triacrylate, pentaerythritol diacrylate, dipentaerythritol diacrylate, dipentaerythritol triacrylate, dipentaerythritol tetraacrylate, dipentaerythritol pentaacrylate, dipentaerythritol hexaacrylate, ethoxylated trimethylolpropane diacrylate, ethoxylated trimethylol propane triacrylate, ethoxylated ditrimethylolpropane diacrylate, ethoxylated ditrimethylolpropane triacrylate, ethoxylated ditrimethylolpropane tetraacrylate, ethoxylated ditrimethylolpropane pentaacrylate, ethoxylated ditrimethylolpropane hexaacrylate, glycerol triacrylate, glycerol diacrylate, propoxylated glycerol diacrylate, propoxylated glycerol triacrylate, ethylene glycol diacrylate, propylene glycol diacrylate, 1,3-butanediol diacrylate, 1,4-butanediol diacrylate, neopentyl glycol diacrylate, diethylene glycol diacrylate, triethylene glycol diacrylate, dipropylene glycol diacrylate, ethoxylated bisphenol A diacrylate, ethoxylated bisphenol F diacrylate, or a combination thereof. The corresponding methacrylates of the foregoing compounds may be used.

Examples of the multifunctional monomer may include pentaerythritol tetraacrylate, pentaerythritol triacrylate, ethoxylated trimethylol propane triacrylate, and ethoxylated bisphenol A diacrylate. The corresponding methacrylates of the foregoing compounds may be used.

In an embodiment, a weight ratio of amounts of the tetraacrylate monomer, triacrylate monomer, and diacrylate monomer may be in a range of about 0.1 to 2:2 to 5:1. For example, the weight ratio of amounts of the tetraacrylate monomer, triacrylate monomer, and diacrylate monomer may be in a range of about 0.2 to about 1.8:1.8 to 4.8:1, about 0.3 to 1.6:about 2.2 to 4.6:1, about 0.3 to 1.4:about 2.4 to 4.6:1, about 0.4 to 1.2:about 2.6 to 4.4:1, about 0.5 to 1.0:about 2.8 to 4.2:1, about 0.6 to about 0.8:3 to 4:1, or about 0.7 to 0.8:about 3.2 to 3.8:1.

Since a strong crosslinking bond may be formed by a combination of acrylate monomers having various numbers of functional groups, the rigidity of the hard coating film obtained after curing of the composition for hard coating according to an embodiment may be improved.

In an embodiment, the monofunctional monomer may include the monofunctional monomer with a hydrophilic group and a monofunctional monomer without a hydrophilic group.

In an embodiment, among the monofunctional monomers, an amount of the monofunctional monomer with a hydrophilic group may be greater than that of the monofunctional optional monomer, in particular a monofunctional monomer without a hydrophilic group.

For example, a weight ratio of the monofunctional monomer with a hydrophilic group and the monofunctional monomer without a hydrophilic group, when present, may be in a range of about 1.5 to 15:1, for example, about 1.6 to 14.5:1, about 1.7 to 14.0:1, about 1.8 to 13.5:1, about 1.9 to 13:1, about 2.0 to 12.5:1, about 2.1 to 12:1, about 2.2 to 11.5:1, about 2.3 to 11:1, about 2.3 to 10.5:1, about 2.4 to 10:1, about 2.4 to 9.5:1, about 2.5 to 9:1, about 2.6 to 8.5:1, about 2.7 to 8:1, about 2.8 to 7.5:1, about 2.9 to 7:1, about 3 to 6.5:1, about 3.1 to 6:1, about 3.2 to 5.5:1, about 3.3 to 5:1, about 3.4 to 4.5:1, or about 3.5 to 4:1.

When the monofunctional monomers include the monofunctional monomer with a hydrophilic group and the monofunctional monomer without a hydrophilic group, a hard coating film having rigid and hydrophilic properties may be formed.

In an embodiment, examples of the hydrophilic group in the monofunctional monomer with a hydrophilic group may include an alcohol group, an alkoxy group, a hydroxyl group, an amino group, a polyether group, or a combination thereof, but embodiments are not limited thereto, and various suitable functional groups capable of imparting hydrophilicity to a molecule may be used.

In an embodiment, the monofunctional monomer with a hydrophilic group may include (meth)acylate containing an alkoxy group and (meth)acrylate containing a hydroxy group.

Examples of the (meth)acrylate including an alkoxy group may include methoxy polyethylene glycol methacrylate, methoxy polyethylene glycol acrylate, methoxy polypropylene glycol methacrylate, methoxy polypropylene glycol acrylate, ethoxy polyethylene glycol methacrylate, ethoxy polyethylene glycol acrylate, ethoxy polypropylene glycol methacrylate, ethoxy polypropylene glycol acrylate, or a combination thereof. Here, the “polyethylene glycol” in the methoxy polypropylene glycol methacrylate has a molecular weight of about 200 or greater. For example, the “polyethylene glycol” has a molecular weight of about 400 or about 600. The methoxy polypropylene glycol methacrylate denotes all methoxy polyethylene glycol methacrylate having a molecular weight of about 200, about 400, or about 600, and other acrylate and methacrylate including polyethylene glycol may be understood in the same manner.

For example, the (meth)acrylate containing a hydroxy group may include hydroxyethyl (meth)acrylate, hydroxypropyl (meth)acrylate, hydroxybutyl (meth)acrylate, or a combination thereof.

In an embodiment, the monofunctional monomer without a hydrophilic group may be acrylate containing a glycidyl group or methacrylate containing a glycidyl group.

In an embodiment, the antibacterial polymer composition may further include an initiator.

In an embodiment, the initiator is a UV-absorbing initiator that absorbs a light at a wavelength of 400 nanometer (nm) or less, and examples of the initiator may include a benzoin ether-based compound, an acetophenone-based compound, an α-ketone-based compound, an oxime-based compound, a benzoin-based compound, and a benzyl-based compound, a benzophenone-based compound, a ketal-based compound, a thioxanthone-based compound, an acylphosphine oxide-based compound, or a combination thereof.

For example, the initiator may be benzoin methyl ether, benzoin ethyl ether, benzoin propyl ether, benzoin isopropyl ether, benzoin isobutyl ether, 2,2-dimethoxy-1,2-diphenylethan-1-one, anisole methyl ether, 2,2-diethoxyacetophenone, 2,2-dimethoxy-2-phenylacetophenone, 1-hydroxycyclohexylphenylketone, 4-phenoxydichloroacetophenone, 4-t-butyldichloroacetophenone, 2-methyl-2-hydroxypropiophenone, 1-[4-(2-hydroxyethyl)phenyl]-2-hydroxy-2-methylpropan-1-one, 1-phenyl-1,1-propanedione-2-(o-ethoxycarbonyl)-oxime, benzoin, benzyl, benzophenone, benzoylbenzoic acid, 3,3′-dimethyl-4-methoxybenzophenone, polyvinylbenzophenone, α-hydroxycyclohexyl phenyl ketone, benzyl dimethyl ketal, thioxanthone, 2-chlorothioxanthone, 2-methylthioxanthone, 2,4-dimethylthioxanthone, isopropyl thioxanthone, 2,4-dichlorothioxanthone, 2,4-diethyl thioxanthone, 2,4-diisopropyl thioxanthone, dodecyl thioxanthone, 2,4,6-trimethylbenzoyl-diphenyl-phosphine oxide, bis(2,4,6-trimethyl benzoyl)-phenylphosphine oxide, or a combination thereof.

An amount of the initiator may be in a range of about 0.5 parts to about 3 parts by weight based on 100 parts by weight of the total amount of the composition, for example, about 0.5 parts to about 2.8 parts, about 0.5 parts to about 2.6 parts, about 0.5 parts to about 2.4 parts, about 0.5 parts to about 2.2 parts, about 0.5 parts to about 2.2 parts, about 0.5 parts to about 2 parts, about 0.5 parts to about 1.8 parts, about 0.5 parts to about 1.6 parts, about 0.5 parts to about 1.4 parts, about 0.5 parts to about 1.2 parts, about 0.5 parts to about 1 parts, or about 0.5 parts to about 0.8 parts by weight based on 100 parts by weight of the total amount of the composition.

In an embodiment, the antibacterial polymer composition may be a solventless composition substantially not including a solvent. Thus, formation of pores due to volatilization of a solvent is suppressed, and formation of a dense antibacterial film may be facilitated.

In an embodiment, to increase applicability, workability, rigidity, hydrophilicity, uniformity, and curing acceleration rate, the antibacterial polymer composition may further include various additives such as a dispersant, a thickener, a leveling agent, and a curing accelerator within a range that does not adversely affect the physical properties of the hard coating film.

### Antibacterial Film

According to an aspect of an embodiment, an antibacterial film may include a polymer including at least one repeating unit represented by Formula 3:

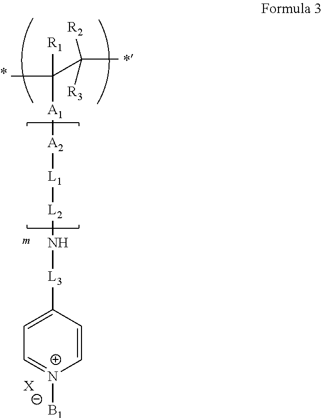

In Formula 3,
A₁ is *—C(═O)—*′, *—C(═O)O—*′, or *—C(═O)NH—*′,
A₂ is *—O—*′, *—S—*′, or *—Se—*′,
L₁, L₂, and L₃ are each independently, a single bond, a substituted or unsubstituted C₁-C₂₀ alkylene group, or a substituted or unsubstituted C₂-C₂₀ alkenylene group,
m is an integer selected from 0 to 3,
B₁ is a substituted or unsubstituted C₁-C₂₀ alkyl group,
R₁ to R₃ are each independently, hydrogen, deuterium, —F, —Cl, —Br, —I, a hydroxyl group, a cyano group, an amino group, a nitro group, a substituted or unsubstituted C₁-C₂₀ alkyl group, a substituted or unsubstituted C₂-C₂₀ alkenyl group, a substituted or unsubstituted C₂-C₂₀ alkynyl group, or a substituted or unsubstituted C₁-C₂₀ alkoxy group,
X is a halogen anion, and
* and *′ each indicate a binding site to an adjacent atom.

The antibacterial film may be obtained by UV-curing the antibacterial polymer composition.

In an embodiment, the antibacterial film may be metal-free.

For example, the antibacterial film may include a metal-free acrylate resin.

In an embodiment, a water contact angle of the antibacterial film may be about 40° or less, and a pencil surface hardness of the antibacterial film at a load of 1 kg may be about 6 H or more. For example, the water contact angle of the antibacterial film may be about 40° to about 0.1°, about 40° to about 1°, about 40° to about 5°, about 40° to about 10° about 40° to about 15°, about 40° to about 20°, about 40° to about 25°, about 40° to about 30°, about 40° to about 35°, or about 40° to about 0.01°. For example, the pencil surface hardness of the antibacterial film at a load of 1 kg may be about 6 H to about 9 H, about 6 H to about 8 H, or about 6 H to about 7 H.

In an embodiment, the acrylate resin may be derived from at least one multifunctional monomer and at least one monofunctional monomer with a hydrophilic group, different from the antibacterial monomer.

In order to obtain a hydrophilic coating film (e.g., having a water contact angle of 40° or less), a metallate and a hydrolyzable silane group-containing compound are mixed and then hydrolyzed, or an anionic hydrophilic group in the form of a metal salt is added to a polymer and cured to form a hydrophilic coating film. However, since the hydrophilic group has a high tendency to be oriented inside the polymer structure due to the high surface energy of the hydrophilic group during the curing process, and the hydrophobic group such as an alkyl group having a low surface energy has a high tendency to be oriented outside the polymer structure, introduction of more metallates has been inevitable to obtain highly hydrophilic groups. Such excessive addition of metallates decreases the rigidity of the final cured coating film, and in particular, when polyethylene glycol (PEG), which is an oligomeric hydrophilic group, is used as a hydrophilic group, the rigidity of the coating layer is severely inhibited due to the soft property of PEG.

As the hydrophilicity and rigidity of the coating film are in a trade-off relationship, simultaneously improving both the hydrophilicity and rigidity of the coating film has been difficult in the art.

The present inventor has found out that when a coating composition is applied using an acrylate-based resin derived from at least one multifunctional monomer and at least one monofunctional monomer with a hydrophilic group, different from the antibacterial monomer, despite not including a metallate, a coating layer having a water contact angle of about 40° and a pencil surface hardness of about 6 H or more at a load of 1 kg may be obtained and that when an antibacterial monomer is added to the coating composition of a predetermined amount, an antibacterial film having antibacterial properties may be prepared without deteriorating the hydrophilicity and rigidity of the film.

When a hydrophobic substrate not coated with the antibacterial film according to an embodiment is contaminated by hydrophobic contaminants and thus needs to be washed using washing water, the washing water may not penetrate an interface between the hydrophobic substrate and the hydrophobic contaminants, which makes it difficult to remove the hydrophobic contaminants from the substrate.

However, since the antibacterial film according to an embodiment has a hydrophobic property, when the surface of the antibacterial film is contaminated by hydrophobic contaminants and thus is washed with washing water, the washing water penetrates an interface between the antibacterial film and the hydrophobic contaminants and breaks the bond between the contaminants and the surface of the antibacterial film, which results in an improved effect of removing the contaminants from the antibacterial film using a small amount of washing water.

In an embodiment, a content ratio of an amount of the multifunctional monomer to an amount of the monofunctional monomer with a hydrophilic group, different from the antibacterial monomer may be in a range of about 1:1 to about 1.5:1, for example, about 1:1 to about 1.4:1, about 1:1 to about 1.3:1, about 1:1 to about 1.2:1, or about 1:1 to about 1.1:1.

When the content ratio of the multifunctional monomer to the monofunctional monomer with a hydrophilic group, different from the antibacterial monomer is within this range, an antibacterial film having rigidity and hydrophilicity may be formed.

In an embodiment, an amount of repeating units derived from the monofunctional monomer with a hydrophilic group may be in a range of about 29 wt % to about 45 wt % in the (meth)acrylate-based resin. For example, the amount of repeating units derived from the monofunctional monomer with a hydrophilic group may be in a range of about 29 wt % to about 45 wt %, about 29 wt % to about 43 wt %, about 29 wt % to about 41 wt %, about 29 wt % to about 39 wt %, about 29 wt % to about 37 wt %, about 29 wt % to about 35 wt %, about 29 wt % to about 33 wt %, about 29 wt % to about 31 wt %, or about 29 wt % to about 30 wt % in the acrylate-based resin.

When the amount of repeating units derived from the monofunctional monomer with a hydrophilic group, different from the antibacterial monomer is within this range, an antibacterial film having a water contact angle of about 40° or less may be formed. When the amount of repeating units derived from the monofunctional monomer with a hydrophilic group, different from the antibacterial monomer is greater than this range, the rigidity of the antibacterial film may be deteriorated, and when the amount of repeating units derived from the monofunctional monomer with a hydrophilic group different from the antibacterial monomer is less than this range, the antibacterial film may not have sufficient hydrophilicity.

In an embodiment, a thickness of the antibacterial film may be in a range of about 100 micrometer (μm) to about 250 μm, for example, about 100 μm to about 230 μm, about 100 μm to about 210 μm, about 100 μm to about 190 μm, about 100 μm to about 170 μm, about 100 μm to about 150 μm, about 100 μm to about 130 μm, or about 100 μm to about 110 μm. When the thickness of the antibacterial film is within this range, the film may sufficiently protect a substrate from contaminants.

### Laminate

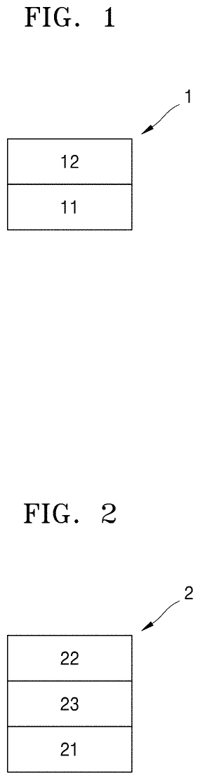

According to an aspect of an embodiment, a laminate is described with reference to FIGS. 1 to 3.

Referring to FIG. 1, a laminate 1 according to an aspect may include a substrate 11; and an antibacterial film 12 described herein on the substrate 11. Here, the antibacterial film 12 may be prepared by curing the antibacterial polymer composition described herein.

When disposed on a substrate, the antibacterial film 12 not only protects the substrate from external contaminants but also has a water contact angle of about 40° or less and a pencil surface hardness of 6 H or greater at a load of 1 kg such that the contaminants on the surface may be easily washed out with water and may kill bacteria by having a pyridinium alkyl group.

In an embodiment, examples of the substrate 11 may include metal plates such as iron, aluminum, copper, or alloys thereof; resin molded products of polyethylene, polypropylene, ethylene-vinyl acetate copolymer, polyamide, polyacrylate, polyester, vinyl chloride resin, vinylidene chloride resin, polycarbonate, polyurethane, or acrylonitrile-butadiene-styrene copolymer; ceramics such as glass; or plastics.

For example, the substrate 11 may be ceramic or plastic.

Referring to FIG. 2, a laminate 2 according to an aspect may include a substrate 21; an antibacterial film 22 on the substrate 21; and a primer layer 23 between the substrate 21 and the antibacterial film 22.

The substrate 21 and the antibacterial film 22 are the same as defined in relation to the substrate and the antibacterial film described herein.

Examples of a coating material for forming the primer layer 23 may include a resin composition having a polyester resin, a polyamide resin, a polyurethane resin, an epoxy resin, a phenol rein, a meth(acrylate) resin, a polyvinyl acetate resin, a polyolefin resin, a copolymer or a modified resin thereof, or a cellulose resin.

In an embodiment, adhesive strength between an antibacterial film and a substrate may be increased without a primer layer by increasing a surface energy of the substrate through corona treatment, flame treatment, or plasma treatment on the substrate.

In an embodiment, a thickness of the primer layer 23 may be in a range of about 2 μm to about 50 μm, for example, about 2 μm to about 45 μm, about 2 μm to about 40 μm, about 2 μm to about 35 μm, about 2 μm to about 30 μm, about 2 μm to about 25 μm, about 2 μm to about 20 μm, about 2 μm to about 15 μm, about 2 μm to about 10 μm, or about 2 μm to about 5 μm.

In an embodiment, the laminate 2 may further include a buffer layer (not shown) between the substrate 21 and the antibacterial film 22. The buffer layer may prevent separation of the substrate 21 and the antibacterial film 22 by absorbing the stress generated according to the difference in surface energy between the substrate 21 and the antibacterial film 22.

For example, when the laminate 2 includes the buffer layer, the buffer layer may be disposed between the substrate 21 and the primer layer 23, between the antibacterial film 22 and the primer layer 23, or both between the substrate 21 and the primer layer 23 and between the antibacterial film 22 and the primer layer 23.

In an embodiment, the laminate 2 may further include at least one functional layer (not shown) on the antibacterial film 22. The functional layer may be disposed on a surface of the antibacterial film 22 and improve durability of the antibacterial film 22.

Referring to FIG. 3, a laminate 3 according to an aspect may include a substrate 31; an antibacterial film 32 on the substrate 31; and a hydrophilic hard coating layer 34 between the substrate 31 and the antibacterial film 32.

The substrate 31 and the antibacterial film 32 are the same as defined in relation to the substrate and the antibacterial film described herein.

The hard coating layer 34 is a cured layer of a mixture of monomers other than an antibacterial monomer in the antibacterial polymer composition described herein and is an acrylate-based hydrophilic hard coating layer having a water contact angle of about 40° or less and a pencil surface hardness of about 6 H or greater at a load of 1 kg.

The hard coating layer 34 is located under the antibacterial film 32 and thus protects the substrate 31 and provides high hydrophilicity and high hardness to the surface of the substrate 31.

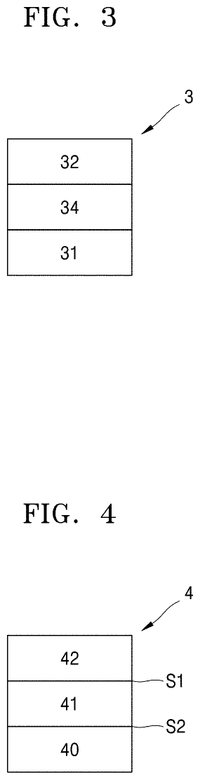

Referring to FIG. 4, a laminate 4 according to an aspect may include a substrate 41; an antibacterial film 42 on a surface S1 of the substrate 41; and an adhesive layer 40 on another surface S2 of the substrate 41.

When a laminate further includes an adhesive layer on another surface of a substrate, the laminate may be applied onto a surface of an article which may be contaminated with external contaminants. Also, when the laminate is applied onto a surface of an article, the surface of an article may be protected from external materials, durability of the article may improve as the surface of the article may easily be washed, and bacterial growth on the surface of the article may be suppressed, which is hygienic as well.

Although not shown in FIG. 4, the laminate 4 may further include a primer layer (not shown) between the substrate 41 and the antibacterial film 42 on the surface S1 of the substrate 41. Here, the primer layer is the same as defined in relation to the primer layer 23 in FIG. 2.

### Article

According to an aspect of an embodiment, an article may include the laminate.

For example, by being provided on an outer surface of the article, the laminate may protect the outer surface of the article from external contaminants, contributes to improvement of durability of the article as it is easily washed with water when the surface is contaminated, and suppresses bacterial growth on the article surface, which may be hygienic as well.

In an embodiment, examples of the article may include glass, mirror, display articles such as a cell phone display, an information board such as a signboard or an advertisement, an electronic device case, an exterior material such as an automobile exterior material, or a bathroom article such as a toilet seat or a washbasin, but embodiments are not limited thereto, and any suitable article of which the surface may be contaminated from the outside and exposed to a humid environment where bacteria may proliferate.

### Method of Preparing Antibacterial Film

According to an aspect of an embodiment, a method of preparing an antibacterial film may include spray-coating an antibacterial polymer composition on a substrate; and forming an antibacterial film on the substrate by irradiating ultraviolet (UV) light to the composition.

In an embodiment, the method may further include forming a primer layer on the substrate before the forming of the antibacterial film.

For example, a primer coating composition may be applied on the substrate before the spray-coating of the antibacterial polymer composition to form the primer layer.

In an embodiment, the primer coating composition may be the resin composition for primer coating described herein.

For example, the substrate may be surface modified prior to the spray-coating of the antibacterial polymer composition. In an embodiment, the surface modification of the substrate may be performed by corona treatment, flame treatment, plasma treatment, or combinations thereof.

In an embodiment, the method may further include forming a hydrophilic hard coating layer on the substrate before the forming of the antibacterial film. In an embodiment, the hydrophilic hard coating layer is a composition from which only the antibacterial monomer is removed from the antibacterial polymer composition described above.

For example, the hydrophilic hard coating layer may be formed by spray-drying a composition for preparing a hydrophilic hard coating layer (hereinafter, also referred to as ‘a composition for hard coating’) at least twice and UV-curing the resultant hydrophilic hard coating layer.

For example, the hydrophilic hard coating layer may be prepared by spraying some of the composition for hard coating as a raw material on the substrate, irradiating UV light thereto, seizing the UV light irradiation before the sprayed composition for hard coating is completely dried, and spraying the remaining composition for hard coating on the incompletely cured hard coating layer, followed by irradiating UV light thereto, thereby preparing a completely cured hard coating layer.

The raw material of the composition for hard coating may be sprayed twice, but embodiments are not limited thereto, and those of skill in the art may appropriately select the number of spraying in consideration of the thickness and physical properties of the hard coating layer. In an embodiment, a primer layer may be provided on the completely cured hard coating layer, and forming an additional hard coating layer may be added to the method.

In an embodiment, after forming the hard coating layer, the antibacterial polymer composition may be spray-coated on the hard coating layer, and UV light may be irradiated thereto to form a cured antibacterial film. Here, the antibacterial film may further have antibacterial properties while maintaining physical properties of a hard coating layer such as a water contact angle of about 40° or less and a pencil surface hardness of about 6 H or more at a load of 1 kg.

In an embodiment, when the substrate is a film, the method may further include providing an adhesive layer on a surface opposite to a surface of the substrate on which the antibacterial film is formed. In an embodiment, the method may further include providing a separation film on a surface of the adhesive layer.

Using the method described herein, an antibacterial adhesive film that may be applied to an article requiring external surface protection and surface sterilization/disinfection may be prepared. In an embodiment, the adhesive layer may be any suitable adhesive in the art such as an acrylate-based adhesive, an epoxy-based adhesive, a novolak-based adhesive, or a combination thereof.

In an embodiment, the method may further include providing a functional layer on the antibacterial film after the forming of the antibacterial film.

The functional layer is a layer for increasing hardness of the antibacterial film, which may be a resin layer including silica or inorganic metal.

In an embodiment, when the method of preparing an antibacterial film is used, the antibacterial polymer composition does not include a solvent, which may omit a drying process, which may result in reduced processing time and saving costs. Also, due to the absence of a solvent, an antibacterial film may be prepared with high uniformity.

### Definition of Substituent

The term “C₁-C₆₀ alkyl group,” as used herein, refers to a linear or branched aliphatic hydrocarbon monovalent group having 1 to 60 carbon atoms, and non-limiting examples thereof include a methyl group, an ethyl group, a propyl group, an isobutyl group, a sec-butyl group, a tert-butyl group, a pentyl group, an iso-amyl group, and a hexyl group. The term “C₁-C₆₀ alkylene group,” as used herein, refers to a divalent group having the same structure as the C₁-C₆₀ alkyl group.

The term “C₂-C₆₀ alkenyl group,” as used herein, refers to a hydrocarbon group formed by substituting at least one carbon-carbon double bond in the middle or at the terminal of the C₂-C₆₀ alkyl group, and examples thereof are an ethenyl group, a propenyl group, and a butenyl group. The term “C₂-C₆₀ alkenylene group,” as used herein, refers to a divalent group having the same structure as the C₂-C₆₀ alkenyl group.

The term “C₂-C₆₀ alkynyl group,” as used herein, refers to a hydrocarbon group formed by substituting at least one carbon-carbon triple bond in the middle or at the terminal of the C₂-C₆₀ alkyl group, and examples thereof are an ethynyl group and a propynyl group. The term “C₂-C₆₀ alkynylene group,” as used herein, refers to a divalent group having the same structure as the C₂-C₆₀ alkynyl group.

The term “C₁-C₆₀ alkoxy group,” as used herein, refers to a monovalent group represented by -OA₁₀₁ (wherein A₁₀₁ is the C₁-C₆₀ alkyl group), and examples thereof include a methoxy group, an ethoxy group, and an isopropyloxy group.

The term “acrylate-based” or “acrylate-containing” as used herein, refers to an acrylate group, a methacrylate group, or a combination thereof.

In case of “substituted” functional groups, the number of carbon atoms in the substituent are not counted in the total number of carbon for that substituent, for example, a C₂₀ alkyl group substituted with a C₂₀ alkoxy group does not mean a C₄₀ alkyl group, similarly, a C₂₀ alkyl group substituted with a C₆ aryl or a phenyl group does not mean a C₂₆ alkyl group.

Hereinafter, an antibacterial monomer, an antibacterial polymer composition, and an antibacterial film and a laminate prepared using the composition according to one or more embodiments will be described in further detail with reference to Examples. The expression “B was used instead of A” used in describing Examples may refer to a molar equivalent of A being identical to a molar equivalent of B.

### EXAMPLES

### Synthesis of Antibacterial Monomer

### Synthesis Example 1

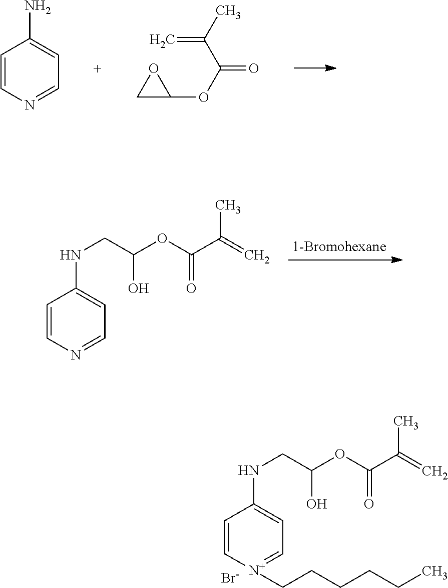

Information and amounts of agents used in Synthesis Example 1 are the same as shown in Table 1.

TABLE 1

| No | Chemical | Abbr. | CAS No. | MW | Amount | Unit | mole | remarks |
|---|---|---|---|---|---|---|---|---|
| 1 | Glycidyl | GMA | 106-91-2 | 142.15 | 10 | g | 0.070 | |
| | Methacrylate | | | | | | | |
| 2 | 4-Amino | APy | 504-24-5 | 94.1 | 5 | g | 0.053 | |
| | Pyridine | | | | | | | |
| 3 | 1-Bromo | BH | 111-25-1 | 165.1 | 12 | g | 0.0727 | |
| | Hexane | | | | | | | |
| 4 | Ethanol | EtOH | 64-17-5 | 46.1 | 30 | ml | | Reaction |
| | | | | | | | | solvent |
| 5 | Ethyl Acetate | EAc | 141-78-6 | 88.1 | 500 | ml | | Precipitation |
| | | | | | | | | solvent |

After completely dissolving 4-amino pyridine (APy) in ethanol (EtOH), glycidyl methacrylate (GMA) was added thereto and reacted in an ice bath. Then, after 2 to 3 hours of the reaction, the mixture was further reacted at room temperature or lower for 4 days. Next, 1-bromo hexane (BH) was added to the reaction solution, and the resultant solution was further reacted at room temperature for 48 hours. Once the reaction was completed, the resulting solution was added to a large amount of an ethyl acetate (EAc) solvent to allow precipitation, and thus a viscous liquid was obtained. This was again dissolved in 30 milliliter (ml) of EtOH and then re-precipitated in EAc to remove unreacted materials. The viscous liquid thus obtained was vacuum-dried at room temperature (25 degrees Celsius or lower) for 48 hours. The dried viscous liquid was subjected to an NMR (nuclear magnetic resonance) measurement using DMSO-D6 as a solvent.

1H-NMR (DMSO-D6); δ=1.05 (t, 3H, CH₃), 1.8 (s, 3H, CH3 C), 2.0 (s, 1H, NH), 3.3 (m, 6H, CH2), 3.7 (s, 2H, CH2 N+), 4.0 (m, 2H, CH2 N), 4.2 (s, 1 H, OH), 5.0 (s, 1 H, CH2 C), 5.5 (s, 1 H, CH2 C), 6.9 (s, 2H, C N), 8.1 (s, 2H, C N) ppm.

### Synthesis Example 2

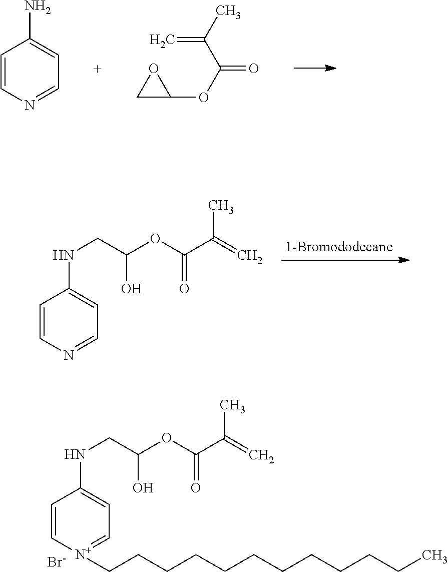

Information and amounts of agents used in Synthesis Example 2 are the same as shown in Table 2.

TABLE 2

| | | | | | | | | mole | |
|---|---|---|---|---|---|---|---|---|---|
| No | Chemical | Abbreviation | CAS No. | MW | Amount | Unit | mole | ratio | remark |
| 1 | Glycidyl | GMA | 106-91-2 | 142.15 | 10 | g | 0.070 | 1.000 | |
| | Methacrylate | | | | | | | | |
| 2 | 4-Amino | APy | 504-24-5 | 94.1 | 5 | g | 0.053 | 0.755 | |
| | Pyridine | | | | | | | | |
| 3 | 1-Bromo | BDD | 143-15-7 | 249.2 | 15 | g | 0.0602 | 0.8556 | |
| | Dodecane | | | | | | | | |
| 4 | Ethanol | EtOH | 64-17-5 | 46.1 | 100 | ml | | | Reaction |
| | | | | | | | | | solvent |
| 5 | Ehyl Acetate | EAc | 141-78-6 | 88.1 | 500 | ml | | | Precipitation |
| | | | | | | | | | solvent |

Bromododecylpyridiniumaminohydroxylethyl methacrylate (BDDPAHEMA) was synthesized in the same manner as in Synthesis Example 1, except that 1-bromododecane was used instead of 1-bromohexane, and the synthesized BDDPAHEMA was subjected to an NMR measurement.

1H-NMR (DMSO-D6); δ=1.1 (t, 3H, CH3), 1.3 (s, 2H, CH2), 1.9 (s, 3H, CH3 C), 2.1 (m, 1H, NH), 3.0 (s, 18H, CH2), 3.6 (m, 2H, CH2 N+), 4.1 (s, 2H, CH2 N), 4.4 (s, 1 H, OH), 5.1 (s, 1 H, CH2 C), 5.6 (s, 1 H, CH2 C), 7.0 (s, 2H, C N), 8.1 (s, 2H, C N) ppm.

### Synthesis Example 3

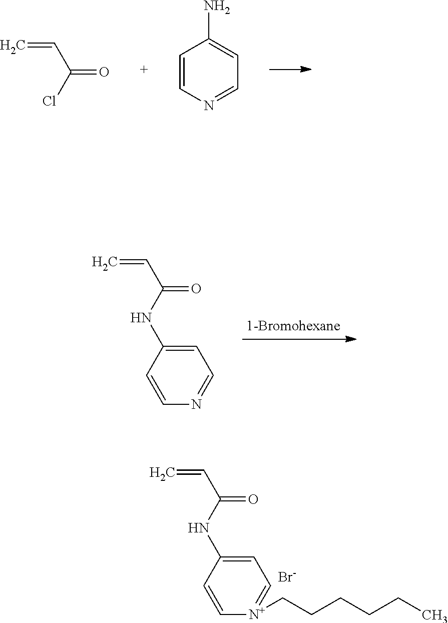

Information and amounts of agents used in Synthesis Example 3 are the same as shown in Table 3.

TABLE 3

| | | | | | | | | mole | |
|---|---|---|---|---|---|---|---|---|---|
| No | Chemical | Abbr. | CAS No. | MW | Amount | Unit | mole | ratio | remark |
| 1 | Acryloyl | AcCl | 814-68-6 | 90.51 | 1.9 | g | 0.021 | 1.000 | |
| | Chloride | | | | | | | | |
| 2 | 4-Amino | APy | 504-24-5 | 94.1 | 2 | g | 0.021 | 1.012 | |
| | Pyridine | | | | | | | | |
| 3 | Triethylamine | TEA | 121-44-8 | 101.19 | 2.5 | g | 0.025 | 1.177 | acid capture |
| 4 | 1-Bromo | BH | 111-25-1 | 165.1 | 4 | g | 0.0242 | 1.1541 | |
| | Hexane | | | | | | | | |
| 5 | Dimethylform- | DMF | 1968-12-02 | 73.09 | 50 | ml | | | Solvent |
| | amide | | | | | | | | |
| 6 | Ethanol | EtOH | 64-17-5 | 46.1 | 100 | ml | | | Reaction |
| | | | | | | | | | solvent |
| 7 | Ethyl Acetate | EAc | 141-78-6 | 88.1 | 500 | ml | | | Precipitation |
| | | | | | | | | | solvent |

APy was completely dissolved using a solvent, DMF, and the solution was mixed with TEA. Then, the mixture was reacted in an ice bath while dropping AcCl. Initially, the mixture was reacted in the ice bath for 2 hours and then reacted at room temperature or lower for 20 hours. Thereafter, the resulting salt was removed by filtration, and EtOH as a solvent was added thereto, followed by mixing with BH to proceed further reaction at room temperature for 20 hours. The reaction solution was precipitated in ethyl acetate to obtain a viscous liquid compound. The compound was dissolved again in 30 ml of EtOH and then re-precipitated in EAc to remove unreacted materials. The viscous liquid thus obtained was vacuum-dried at room temperature (25 degrees Celsius or lower) for 48 hours. The dried viscous liquid was subjected to an NMR (DMSO-D6) measurement.

1H-NMR (DMSO-D6); δ=1.0 (t, 3H, CH3), 1.2 (s, 2H, CH2), 2.7 (s, 6H, CH2), 3.4 (m, 2H, CH2 N+), 5.5 (s, 1 H, CH2), 6.1 (s, 1 H, CH2), 6.4 (s, 1 H, CH), 7.1 (s, 2H, C N), 8.1 (s, 2H, C N), 9.1 (s, 1H, NH) ppm.

### Preparation of Antibacterial Polymer Composition

### Preparation Examples 1 to 8

As shown in Table 4, an antibacterial monomer, a multifunctional monomer, a monofunctional monomer with a hydrophilic group, and an initiator were mixed to prepare an antibacterial polymer composition.

### Evaluation Example 1

Amounts of the multifunctional monomer, amounts of the monofunctional monomer, ratios of the multifunctional monomer/monofunctional monomer, amounts of the hydrophilic monomer, content ratios of the hydrophilic monomer, and content ratios of the antibacterial agent in the antibacterial polymer compositions prepared in Preparation Examples 1 to 8 were calculated and are shown in Table 4.

TABLE 4

| | | Composition (g) | | | | | | | |
|---|---|---|---|---|---|---|---|---|---|
| | | Prep. | Prep. | Prep. | Prep. | Prep. | Prep. | Prep. | Prep. |
| | Raw material | Ex. 1 | Ex. 2 | Ex. 3 | Ex. 4 | Ex. 5 | Ex. 6 | Ex. 7 | Ex. 8 |
| Multifunctional | Pentaerythritol tetraacrylate | 12 | 12 | 12 | 12 | 12 | 12 | 12 | 12 |
| monomer | Pentaerythritol triacrylate | 5 | 5 | 5 | 5 | 5 | 5 | 5 | 5 |
| | Ethoxylated (EO15) trimethylol | 25 | 25 | 25 | 25 | 25 | 25 | 25 | 25 |
| | propane triacrylate | | | | | | | | |
| | Bisphenol A ethoxylated (EO10) | 7 | 7 | 7 | 7 | 7 | 7 | 7 | 7 |
| | diacrylate | | | | | | | | |
| Total | Methoxy PEG600 methacrylate | 10 | 10 | 10 | 10 | 10 | 10 | 10 | 10 |
| Monofunctional | 2-Hydroxyethyl acrylate | 25 | 25 | 25 | 25 | 25 | 25 | 25 | 25 |
| monomer | Glycidyl methacrylate | 3 | 3 | 3 | 3 | 3 | 3 | 3 | 3 |
| | Total | 87 | 87 | 87 | 87 | 87 | 87 | 87 | 87 |
| Antibacterial | Bromohexylpyridinium | 2.18 | 1.09 | 0.54 | 0.27 | 0.14 | 0 | 0.07 | 0 |
| agent | aminohydroxyethyl methacrylate | | | | | | | | |
| | Bromohexyl-3- | 0 | 0 | 0 | 0 | 0 | 0 | 0 | 1.09 |
| | (methacrylamidomethyl) | | | | | | | | |
| | pyridinium | | | | | | | | |
| Initiator | Darocur 1173 | 2.5 | 2.5 | 2.5 | 2.5 | 2.5 | 2.5 | 2.5 | 2.5 |
| | (2-Hydroxy-2- | | | | | | | | |
| | methylpropiophenone) | | | | | | | | |

| | | | | | | | | |
|---|---|---|---|---|---|---|---|---|
| Amount of multifunctional monomer | 49 | 31 | 49 | 49 | 49 | 49 | 49 | 49 |
| Amount of total monofunctional monomer | 38 | 28 | 38 | 38 | 38 | 38 | 38 | 38 |
| Ratio of multifunctional/total monofunctional monomer | 1.29 | 1.11 | 1.29 | 1.29 | 1.29 | 1.29 | 1.29 | 1.29 |
| Amount of monofunctional monomer with a hydrophilic | 35.00 | 25.00 | 35.00 | 35.00 | 35.00 | 35.00 | 35.00 | 35.00 |
| group | | | | | | | | |
| Content ratio of monofunctional monomer with a | 40.2% | 42.4% | 40.2% | 40.2% | 40.2% | 40.2% | 40.2% | 40.2% |
| hydrophilic group | | | | | | | | |
| Amount of antibacterial agent | 2.500% | 1.250% | 0.625% | 0.3125% | 0.1563% | 0 | 0.0781% | 1.250% |

### Example 1

The antibacterial polymer composition prepared in Preparation Example 1 was spray-coated on an ABS resin sheet, and UV light irradiation was performed thereon at a light intensity of 100 millijoule per centimeter (mJ/cm) for 20 seconds using a high-pressure mercury lamp to form a cured antibacterial film, thereby obtaining a laminate. A thickness of the obtained laminate is shown in Table 6.

### Examples 2 to 5

Laminates were each obtained in the same manner as in Example 1, except that the compositions prepared in Preparation Examples 2 to 5 were respectively used instead of the antibacterial polymer composition prepared in Preparation Example 1. Thicknesses of the obtained laminates are shown in Table 6.

### Comparative Examples 1 to 2

Laminates were each obtained in the same manner as in Example 1, except that the compositions prepared in Preparation Examples 6 and 7 were respectively used instead of the antibacterial polymer composition prepared in Preparation Example 1. Thicknesses of the obtained laminates are shown in Table 6.

### Evaluation Example 2: Antibacterial Test

The laminates of Examples 1 to 5 and Comparative Examples 1 to 3 were each prepared in a size of 5 centimeter (cm)×5 cm, and then the surface was irradiated with UV (254 nm) for 2 hours to sterilize, thereby preparing films.

S. aureus (Staphylococcus aureus ATCC 6538P) and E. coli (Escherichia coli ATCC 8739) were each inoculated in an amount of 0.4 ml on the surface of each of the films, and the antibacterial performance was tested according to Japanese Industrial Standard (JIS) Z 2801-2010.

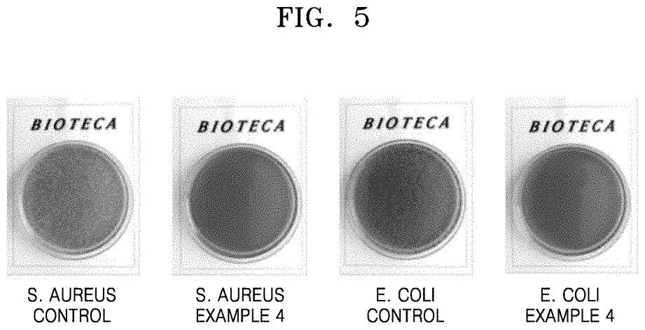

The results are shown in Table 4, and images of the antibacterial performance test performed on Example 4 are shown in FIG. 5.

TABLE 5

| | | | Antibacterial performance | |
|---|---|---|---|---|
| | Amount of antibacterial | | (Bacterial removal rate %) | |
| | monomer | | S. aureus | E. coli |
| Example 1 | 2.5 | wt % | 99.9 | 99.9 |
| Example 2 | 1.25 | wt % | 99.9 | 99.9 |
| Example 3 | 0.625 | wt % | 99.9 | 99.9 |
| Example 4 | 0.3125 | wt % | 99.9 | 99.9 |
| Example 5 | 0.1563 | wt % | 99.9 | 99.9 |
| Comparative | 0% | | 46.7 | 34.5 |
| Example 1 | | | | |
| Comparative | 0.0781 | wt % | 67.3 | 53.5 |
| Example 2 | | | | |
| Comparative | 1.25 | wt % | 16.2 | 15.0 |
| Example 3 | | | | |

Referring to Table 5 and FIG. 5, the antibacterial film, which was cured using an antibacterial polymer composition including an antibacterial agent of about 0.1 wt % or more, had a drastic effect of killing 99.9% of both E. coli and S. aureus, which are microorganisms.

### Evaluation Example 3: Measurement of Water Contact Angle

0.3 microliter (μL) of water droplets were allowed to settle on the surface of each of the laminates obtained in Examples 1 to 5 and Comparative Examples 1 and 2 at room temperature (25° C.) using the Sessile Drop method, and the water contact angle was measured three times using a method of measuring an angle between the water droplet and the surface. The results are shown in Table 6.

### Evaluation Example 4: Measurement of Pencil Hardness

According to the ISO 15184 measurement method, pencil hardness of each of the laminates obtained from Examples 1 to 5 and Comparative Examples 1 and 2 was measured using a Mitsubishi UNI pencil under the loads of 500 gram (g) and 1 kilogram (kg), and the results are shown in Table 6.

TABLE 6

| | | | | | | | Comparative | Comparative |
|---|---|---|---|---|---|---|---|---|
| | | Example 1 | Example 2 | Example 3 | Example 4 | Example 5 | Example 1 | Example 2 |
| Contact | First | 38.9 | 37.2 | 36.4 | 35.2 | 35.3 | 35.1 | 34.4 |
| angle | Second | 37.6 | 35.4 | 35.8 | 35.4 | 34.3 | 35.8 | 33.4 |
| | Third | 36.3 | 34.9 | 35.2 | 33.2 | 34.7 | 33 | 33.7 |
| | Average | 37.6 | 35.8 | 35.8 | 34.6 | 34.8 | 34.6 | 33.8 |
| Pencil | 500 g of | 9H | 9H | 9H | 9H | 9H | 9H | 9H |
| hardness | load | | | | | | | |
| | 1 kg of | 7H | 8H | 8H | 8H | 7H | 8H | 8H |
| | load | | | | | | | |
| Thickness | μm | 150-180 | 170-200 | 150-200 | 150-190 | 150-180 | 150-200 | 150-200 |

As shown in Table 6, the amount of the multifunctional monomer was greater than that of the monofunctional monomer, and when the antibacterial polymer composition including the monofunctional monomer with a hydrophilic group at an amount in a range of about 29 wt % to about 45 wt % based on the total amount of the composition is used to prepare an antibacterial film, it was confirmed that the antibacterial film thus obtained had a water contact angle of about 40° or less and a pencil surface hardness of about 6 H or more at a load of 1 kg.

Also, when the amount of the antibacterial monomer was about 0.1 wt % or more, it was confirmed that the antibacterial film had a function as a coating layer having high hydrophilicity and high rigidity as compared to when an antibacterial agent was not added (Comparative Example 1).

Therefore, it was confirmed that when an antibacterial monomer is used with a multifunctional monomer and a monofunctional monomer with a hydrophilic group, an antibacterial film having high hydrophilicity, high rigidity, and antibacterial properties may be obtained.

According to one or more embodiments, an antibacterial monomer may be formed as a coating layer through a UV-curing process on a surface of a substrate. The antibacterial monomer is not eluted as the antibacterial monomer has a double bond that covalently binds with the coating layer, and bacteria is killed by a pyridinium cation group and an alkyl group of the antibacterial monomer, which thus provides antibacterial properties to the coating layer.

Also, since the antibacterial monomer is mixed with a hydrophilic, highly rigid acrylic resin to form a coating layer, antibacterial properties may be imparted to the hydrophilic and highly rigid film.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the present detailed description as defined by the following claims.

What is claimed is:

1. An antibacterial monomer represented by Formula 1: 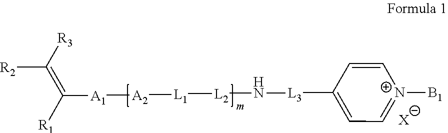
wherein, in Formula 1,
A₁ is *—C(═O)—*′, *—C(═O)O—*′, or *—C(═O)NH—*′,
A₂ is *—O—*′, *—S—*′, or *—Se—*′,
L₁, L₂, and L₃ are each independently, a single bond, a substituted or unsubstituted C₁-C₂₀ alkylene group, or a substituted or unsubstituted C₂-C₂₀ alkenylene group,
m is an integer selected from 0 to 3,
B₁ is a substituted or unsubstituted C₁-C₂₀ alkyl group,
R₁ to R₃ are each independently, hydrogen, deuterium, —F, —Cl, —Br, —I, a hydroxyl group, a cyano group, an amino group, a nitro group, a substituted or unsubstituted C₁-C₂₀ alkyl group, a substituted or unsubstituted C₂-C₂₀ alkenyl group, a substituted or unsubstituted C₂-C₂₀ alkynyl group, or a substituted or unsubstituted C₁-C₂₀ alkoxy group,
X is a halogen anion, and
* and *′ each indicate a binding site to an adjacent atom.

2. The antibacterial monomer of claim 1,
wherein m is 0 or 1,
when m is 0, A₁ is *—C(═O)—*′, and
when m is 1, A₁ is *—C(═O)—*′, and A₂ is *—O—*′.

3. The antibacterial monomer of claim 1,
wherein L₁, L₂, and L₃ are each independently a single bond or a group represented by Formula 2: 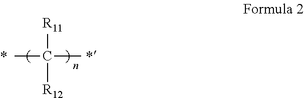
wherein, in Formula 2,
n is an integer selected from 1 to 20, and
R₁₁ and R₁₂ are each independently hydrogen, deuterium, —F, —Cl, —Br, —I, a hydroxyl group, a cyano group, an amino group, a nitro group, a polyether group, a C₁-C₂₀ alkyl group, or a C₁-C₂₀ alkoxy group.

4. The antibacterial monomer of claim 1,
wherein L₁, L₂, and L₃ are each independently a single bond or a group of Formulae 2-1 to 2-10: 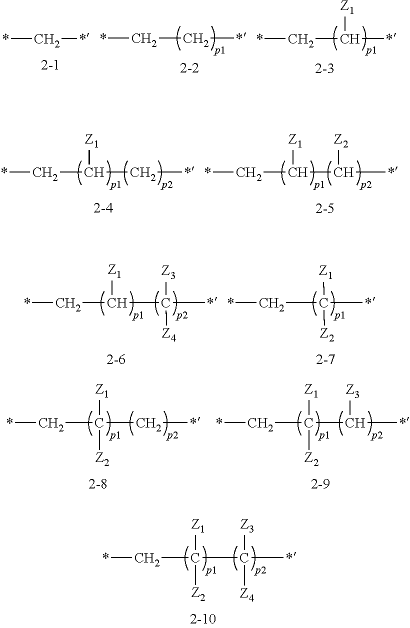
wherein, in Formulae 2-1 to 2-10,
p1 is an integer selected from 1 to 19,
p2 is an integer selected from 1 to 18,
when p1 and p2 are concurrently present, the sum of p1 and p2 is an integer of 19 or less,
Z₁ to Z₄ are each independently a hydroxyl group, an amino group, a nitro group, or a polyether group, and
* and *′ each indicate a binding site to an adjacent atom.

5. The antibacterial monomer of claim 3,
wherein L₃ is a single bond.

6. The antibacterial monomer of claim 1,
wherein B₁ is a straight-chain C₁-C₂₀ alkyl group.

7. The antibacterial monomer of claim 1,
wherein R₁ to R₃ are each independently hydrogen, deuterium, —F, —Cl, —Br, —I, a hydroxyl group, a cyano group, an amino group, a nitro group, a C₁-C₂₀ alkyl group, or a C₁-C₂₀ alkoxy group; or
a C₁-C₂₀ alkyl group or a C₁-C₂₀ alkoxy group, each independently substituted with at least one of deuterium, —F, —Cl, —Br, —I, a hydroxyl group, a cyano group, an amino group, a nitro group, or a C₁-C₂₀ alkoxy group.

8. The antibacterial monomer of claim 1,
wherein R₁ is:
hydrogen, deuterium, —F, —Cl, —Br, —I, a hydroxyl group, a cyano group, an amino group, a nitro group, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group; or
a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group, each independently substituted with at least one of deuterium, —F, —Cl, —Br, —I, a hydroxyl group, a cyano group, an amino group, a nitro group, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group.

9. The antibacterial monomer of claim 1,
wherein R₂ and R₃ are each independently
hydrogen, a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group.

10. The antibacterial monomer of claim 1,
wherein the antibacterial monomer is Compound 1, Compound 2, or Compound 3:

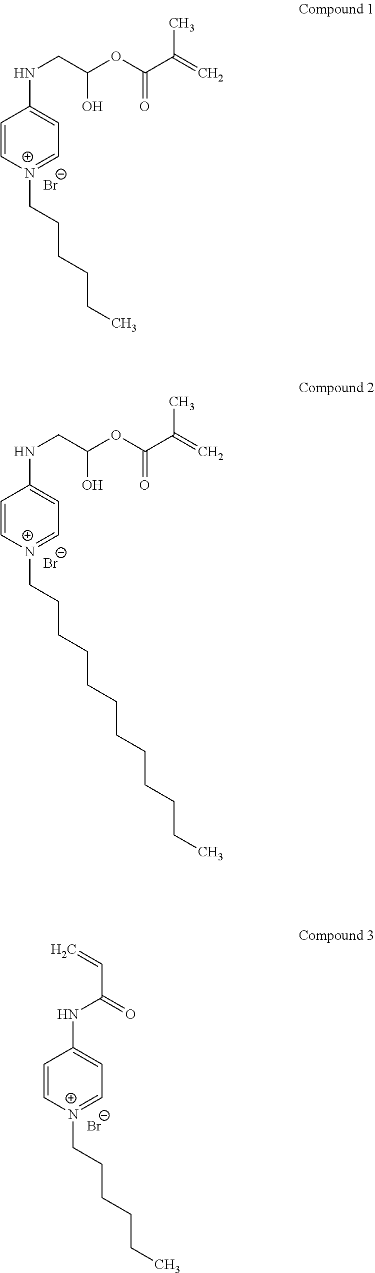 [SMILES: C=C(C)C(=O)OC(O)CNc1cc[n+](CCCCCC)cc1 ; [Br-] ; C=C(C)C(=O)OC(O)CNc1cc[n+](CCCCCCCCCCCC)cc1 ; [Br-] ; C=CC(=O)Nc1cc[n+](CCCCCC)cc1 ; [Br-]]

11. An antibacterial polymer composition comprising:
the antibacterial monomer of claim 1;
a multifunctional monomer; and
a monofunctional monomer with a hydrophilic group, different from the antibacterial monomer.

12. The antibacterial polymer composition of claim 11,
wherein an amount of the antibacterial monomer is about 0.1 weight % or more based on the total amount of monomers in the antibacterial polymer composition.

13. The antibacterial polymer composition of claim 11,
wherein the multifunctional monomer is an (meth)acrylate multifunctional monomer, and
the monofunctional monomer is an (meth)acrylate monofunctional monomer.

14. The antibacterial polymer composition of claim 11,
wherein an amount of the multifunctional monomer is greater than an amount of the monofunctional monomer.

15. The antibacterial polymer composition of claim 11,
wherein a content ratio of the multifunctional monomer to the monofunctional monomer is in a range of about 1:1 to about 1.5:1.

16. The antibacterial polymer composition of claim 11,
wherein an amount of the monofunctional monomer with a hydrophilic group is in a range of about 29 weight % to about 45 weight % based on the total amount of the composition.

17. An antibacterial film comprising a polymer comprising at least one repeating unit represented by Formula 3: 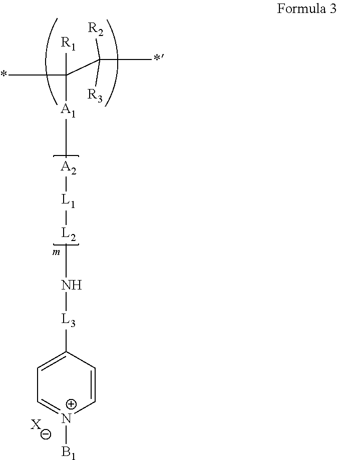
wherein, in Formula 3,
A₁ is *—C(═O)—*′, *—C(═O)O—*′, or *—C(═O)NH—*′,
A₂ is *—O—*′, *—S—*′, or *—Se—*′,
L₁, L₂, and L₃ are each independently, a single bond, a substituted or unsubstituted C₁-C₂₀ alkylene group, or a substituted or unsubstituted C₂-C₂₀ alkenylene group,
m is an integer selected from 0 to 3,
B₁ is a substituted or unsubstituted C₁-C₂₀ alkyl group,
R₁ to R₃ are each independently, hydrogen, deuterium, —F, —Cl, —Br, —I, a hydroxyl group, a cyano group, an amino group, a nitro group, a substituted or unsubstituted C₁-C₂₀ alkyl group, a substituted or unsubstituted C₂-C₂₀ alkenyl group, a substituted or unsubstituted C₂-C₂₀ alkynyl group, or a substituted or unsubstituted C₁-C₂₀ alkoxy group,
X is a halogen anion, and
* and *′ each indicate a binding site to an adjacent atom.

18. The antibacterial film of claim 17,
wherein the antibacterial film is metal-free.

19. The antibacterial film of claim 17,
wherein a water contact angle of the antibacterial film is about 40° or less, and a pencil surface hardness of the antibacterial film at a load of 1 kilogram is about 6 H or more.

20. An article comprising the antibacterial film of claim 17.
